(19) United States
(12) Patent Application Publication
(10) Pub. No.: US 2013/0011430 A1
(43) Pub. Date: Jan. 10, 2013

(54) HUMAN CHORIONIC GONADOTROPIN (hCG) BASED VACCINE FOR PREVENTION AND TREATMENT OF CANCER

(76) Inventors: Om Singh, New Delhi (IN); Rahul Pal, New Delhi (IN); Priyanka Khare, New Delhi (IN); Anjali Bose, New Delhi (IN); Sandhya Singh, New Delhi (IN)

(21) Appl. No.: 13/290,839

(22) Filed: Nov. 7, 2011

Related U.S. Application Data
(63) Continuation of application No. PCT/IN2011/000009, filed on Jan. 6, 2011.

(30) Foreign Application Priority Data
Jan. 8, 2010 (IN) 47/DEL/2010

(51) Int. Cl. A61K 39/385; A61P 35/00

(52) U.S. Cl. 424/195.11

(57) ABSTRACT

The present invention is directed to a vaccine and a pharmaceutical composition comprising hCG and Mycobacterium w suitable for administration to a subject in need thereof for the prevention and/or treatment of cancer. The compositions when administered to a subject in need thereof results in enhanced immunogenicity and prevention against cancer. Furthermore, the compositions of the present invention, when administered to a subject in need thereof, result in inhibition of tumor growth.

### RELATED APPLICATIONS

The present patent document is a continuation of PCT Application Serial No. PCT/IN2011/000009, filed Jan. 6, 2011, designating the United States and published in English, which claims priority to Indian Patent Application Serial No. 47/DEL/2010, filed Jan. 8, 2010, the entire contents of each are incorporated herein by reference.

### BACKGROUND

1. Technical Field Text

The present invention relates to human chorionic gonadotropin (hCG) based vaccines for prevention and treatment of cancer.

2. Background Information

Human chorionic gonadotropin (hCG) is made by the pre-implantation embryo and subsequently by the placenta. It is becoming increasingly clear that a large variety of cancers also unexpectedly synthesize hCG. Its production has been linked to radio- and chemo-resistance as well as to poor patient prognosis. Interestingly, in a few instances, the molecule and/or its subunits have been shown to act as autocrine growth factors. Anti-hCG vaccination strategies for the control of human malignancies thus assume significance. Indeed, even a sub-optimal vaccine targeting hCG has demonstrated some clinical benefit against colorectal cancer. It is believed that lack of immunogenicity as well as the relatively poor affinity of the generated antibodies may have compromised efficacy.

### SUMMARY

One aspect of the present invention is to ascertain the presence of hCG subunits in the human colorectal cell line (COLO205) and the human lung cancer cell line (ChaGo) by semi-quantitative polymerase chain reaction (PCR). Another objective is to ascertain the presence of hCG subunits (both on the cell surface and the cytoplasm) in the two cell lines by indirect immune-fluorescence analysis.

Another aspect of the present invention is to demonstrate the inhibitory effects of anti-hCG antibodies on the growth of COLO 205 and ChaGO cells in culture. The invention would also seek to demonstrate the inhibitory effects of anti-hCG antibodies on hCG-induced Vascular Endothelial Growth Factor (VEGF), Interleukin-8 (IL-8) and Matrix Metalloprotease (MMP) 2 and MMP-9 from tumor cells. These factors have been shown to be critically involved in the growth and metastasis of tumors of many lineages.

Another aspect of the present invention is to demonstrate the invasion-inducing properties of hCG and the ability of anti-hCG antibodies to inhibit invasion into a collagen and laminin based substrate.

Yet another aspect of the present invention is to demonstrate the inhibitory effects of anti-hCG antibodies on the growth of COLO 205 and ChaGO cells implanted in nude mice.

Yet another aspect of the present invention is to demonstrate the benefit of including Mycobacterium w in hCG vaccine formulations (in terms of the antibody titres and neutralizing capabilities of the antibodies generated) in mouse strains traditionally considered low responders to the traditional vaccine formulation.

Yet another aspect of the present invention is to demonstrate the effects of active immunization against hCG on the growth of a model murine lung cancer LL2. This tumor has been traditionally employed as a surrogate to assess the efficacy of anti-hCG vaccination strategies. The objective would include demonstration of the beneficial effects of both pre- and concurrent immunization vis-à-vis tumor implantation, both in terms of tumor volumes and survival statistics. A further objective is to demonstrate the additional benefit of the supplementation of Mycobacterium w on these parameters.

Yet another aspect of the present invention is to provide a therapeutic and/or prophylactic cancer vaccine composition for cancer therapy, the composition comprising βhCG, Mycobacterium w and a pharmaceutically acceptable excipient.

In another embodiment, the invention relates to a vaccine composition for cancer therapy and/or cancer prophylaxis, the composition comprising hCG conjugate, Mycobacterium w and a pharmaceutically acceptable excipient. The cancer may be human lung cancer, colon cancer, testicular cancer, ovarian cancer, bladder cancer, renal cancer, prostate cancer, head and neck cancer or colorectal cancer. Preferably, the hCG is βhCG. The hCG may be conjugated with tetanus toxoid, diphtheria toxoid, or T helper peptide. The T helper peptide may be derived from a pathogen protein selected from the group consisting of tetanus toxin, Plasmodium falciparum circumsporozoite protein, respiratory syncytial virus 1A protein, measles virus fusion protein, influenza virus hemagglutinin, and hepatitis B surface antigen. Preferably, the Mycobacterium w is killed by physical method of heat radiation. The vaccine composition may optionally comprise an adjuvant selected from the group consisting of aluminum hydroxide, Incomplete Fruend's Adjuvant, endotoxin based adjuvants, mineral oil, mineral oil and surfactant, Ribi adjuvant, Titer-max, syntax adjuvant formulation, aluminum salt adjuvant, nitrocellulose adsorbed antigen, immune stimulating complexes, Gebru adjuvant, super carrier, elvax 40w, L-tyrosine, monatanide (manide-oleate compound), Adju prime, Squalene, Sodium phthalyl lipopoly saccharide, calcium phosphate, saponin, and muramyl dipeptide (MDP). The composition may be administered to a subject in need thereof at a dose ranging from 10 to 500 million Mycobacterium w and 1 μg to 50 μg hCG; from 10 to 500 million Mycobacterium w and 50 μg to 500 μg hCG; from 100 to 200 million Mycobacterium w and 100 to 200 μg hCG; or at a dose of 100 million Mycobacterium w. and 100 μg hCG. The vaccine composition may be administered to a subject in need thereof in combination with a therapy selected from the group consisting of radiation therapy and chemotherapy. The vaccine composition may be administered by parental route, intramuscular subcutaneous, or intradermal route.

In another embodiment, the invention is directed to a vaccine composition, comprising hCG conjugate, Mycobacterium w and a pharmaceutically acceptable excipient, wherein the vaccine composition stimulates a non-specific immune response in a subject to restrict tumor growth in the subject.

In a further embodiment, the present invention is directed to a method of treating cancer in a subject, comprising administering a vaccine composition comprising hCG conjugate, Mycobacterium w and a pharmaceutically acceptable excipient to the subject. The cancer may be human lung cancer, colon cancer, testicular cancer, ovarian cancer, bladder cancer, renal cancer, prostate cancer, head and neck cancer or colorectal cancer. The method may further include a step of administering an adjuvant selected from the group consisting of aluminum hydroxide, Incomplete Fruend's Adjuvant, endotoxin based adjuvants, mineral oil, mineral oil and surfactant, Ribi adjuvant, Titer-max, syntax adjuvant formulation, aluminum salt adjuvant, nitrocellulose adsorbed antigen, immune stimulating complexes, Gebru adjuvant, super carrier, elvax 40w, L-tyrosine, monatanide (manide-oleate compound), Adju prime, Squalene, Sodium phthalyl lipopoly saccharide, calcium phosphate, saponin, and muramyl dipeptide (MDP). In the method, the vaccine composition may non-specifically stimulate an immune response in the subject to independently restrict tumor growth.

### DETAILED DESCRIPTION OF THE DRAWINGS AND THE PRESENTLY PREFERRED EMBODIMENTS

The present invention provides human chorionic gonadotropin (hCG) based vaccine composition for treatment and or prevention of cancer. The present invention provides use of anti-hCG vaccination strategies for the control of cancer which secrete or are sensitive to hCG and which may utilize it as an autocrine growth factor. The supplemental use of Mycobacterium w in active immunization schedules is demonstrated to provide dual benefits; the enhancement of anti-hCG titres results from the inclusion of Mycobacterium w, and its non-specific stimulation of the immune stimulation independently restricts tumor growth.

The present invention provides control of human cancers by anti-hCG antibodies. This invention further demonstrates the significant adjuvant effects of Mycobacterium w, (reflected in the significant enhancement of anti-hCG antibody levels) when included in hCG vaccine formulations. The additional, non-specific immunostimulatory activity of Mycobacterium w acts in synergy with anti-hCG antibodies to impair tumor growth.

The Mycobacterium w used in the vaccine composition disclosed in the present invention is a non-pathogenic, fast growing cultivable Mycobacterium belonging to Runyons group IV class of Mycobacteria.

Using both in vitro and in vivo models as well as active and passive immunization approaches, the present invention describes the control of human cancers by anti-hCG antibodies. Mycobacteria, principally BCG, have been used in the treatment of renal cancer; effects are thought to be mainly due to non-specific immune stimulatory events. The present invention further demonstrates the significant adjuvant effects of Mycobacterium w, (reflected in the significant enhancement of anti-hCG antibody levels) when included in hCG vaccine formulations. The additional, non-specific immunostimulatory of Mycobacterium w acts in synergy with anti-hCG antibodies to impair tumor growth.

Human chorionic gonadotropin (hCG) is known to be ectopically expressed by a variety of trophoblastic and non-trophoblastic cancers and can act as an autocrine growth promoter. The presence of hCG (or its subunits) is associated with increased invasiveness, chemo- and radio-resistance and poor prognosis. The present study focused on assessment of immunogenicity and evaluation of efficacy of anti-hCG vaccination on hCG-secreting tumors by employing Mycobacterium was an adjuvant along with a prototypic vaccine formulation (βhCG-TT adsorbed on aluminum hydroxide). Incorporation of Mycobacterium w along with the hCG vaccine formulation led to significantly enhanced immunogenicity in mice of diverse genetic background (H-2^{d}, H-2^{b}, H-2^{k}, H-2q).

Surprisingly, significant increases in IgG2a and IgG2b antibody levels were observed, providing insight into the differences in the T cell responses induced by mycobacterium. Mycobacterium w-supplemented formulations elicited higher titres of biologically active antibodies which more potently inhibited receptor-hCG interaction; in all instances, antibodies exhibited high affinity (≅10¹⁰ M⁻¹) for hCG. Further, elicited antibodies were reactive towards the surface of human colorectal carcinoma and non-small cell lung carcinoma cell lines. Anti-hCG antibodies were capable of inducing cytocidal effects even in the absence of complement. Taken together, our findings suggest that inclusion of Mycobacterium w in anti-hCG vaccine formulations can enhance adjuvanticity (even in murine strains traditionally considered hypo-responsive) for the generation of specific, therapeutic antibodies, along with providing the expected up-modulation of general immunity via non-specific mechanisms.

The presence mRNA for the hCG subunits in the human colorectal cancer cell line COLO 205 and the human lung cancer ChaGo was demonstrated by semi-quantitative PCR. Anti-hCG antibodies bound the tumor cells on the surface as well as in intra-cellular compartments in indirect immunofluorescence assays. Specificity of binding was ascertained by competitive studies.

While hCG induced enhanced growth in both cell lines in vitro, anti-hCG antibodies neutralized these effects. hCG induced the expression of VEGF and IL8 as ascertained by semi-quantitative PCR for mRNA. ELISAs revealed significant increases in protein levels as well. Zymogram analysis revealed hCG induced up-modulation in the levels of active MMP-2 and MMP-9. All these effects were effectively neutralized by the addition of anti-hCG antibodies.

hCG was shown to increase the invasiveness of COLO 205, ChaGo and LL2 cells in vitro, using a collagen and laminin based synthetic substrate. Anti-hCG antibodies could effectively negate these effects, whereas control antibodies had no effect.

COLO 205, ChaGo and LL2 cells were independently implanted in nude mice. Concurrent parenteral administration of anti-hCG antibodies, while not decreasing tumor incidence, significantly reduced tumor volumes in all animals. Control antibodies had no effect.

Co-administration of Mycobacterium w with the hCG vaccine formulation (a stoichiometrically controlled conjugate of βhCG and tetanus toxoid, adsorbed on aluminum hydroxide) resulted in a significant elevation of anti-hCG antibody titres in mice. Antibodies were of high affinity and neutralized the biological actions of hCG.

LL2 (murine lung tumor cells) were subcutaneously implanted into syngeneic C57BL/6 mice. Animals were previously or concurrently immunized with the prototypic vaccine, with or without additional supplementation with Mycobacterium w. Some animals received Mycobacterium w alone. While all immunized animals demonstrated decreased tumor growth, the most significant effects were seen in animals immunized with both the prototypic vaccine and Mycobacterium w. Decreases in tumor incidence as well as size were noted, and survival statistics were significantly enhanced.

All non-vaccinated (control) mice developed tumors 3 weeks after implantation. It was found that immunization with βhCG-TT vaccine or with Mycobacterium w prevented tumor development in 1 of 11 (9.1%) and 4 of 11 (36.4%) mice respectively, whereas unexpectedly in a group immunized with the combination of βhCG-TT conjugate and Mycobacterium w (βhCG-TT+Mycobacterium w), 10 of 12 (83.3%) mice did not develop tumor. Thus the level of efficacy achieved in terms of tumor prevention with the combination of βhCG-TT conjugate and Mycobacterium w is not only significantly higher than the efficacy achieved by βhCG-TT conjugate and Mycobacterium w when individually immunized, but also significantly greater than the theoretical expected efficacy of the combination, which was calculated to be 42.3%. Analysis of the tumor volumes further confirms the superior anti-tumor effects of co-immunization with βhCG-TT conjugate and Mycobacterium w. As against the average volume of tumors which developed in non-vaccinated mice (11.9 cm³), the average tumor volume in the βhCG-TT immunized group of mice was 4.2 cm³ and in and in the Mycobacterium w immunized group was 2.9 cm³, indicating tumor growth inhibiting efficacies of 64.3% and 75.6% respectively. While the theoretical expected tumor growth-inhibiting effect in mice immunized with the combination of βhCG-TT conjugate and Mycobacterium w was 91.4%, the actual tumor growth-inhibiting effect observed in these animals was 98.2% which is significantly higher than the expected efficacy. Average tumor volumes were 0.22 cm³ which is significant reduction in the tumor size as compared to those in mice individually immunized with βhCG-TT conjugate or Mycobacterium w. Thus it clear that the combination of βhCG-TT conjugate and Mycobacterium w shows synergistic anti-tumor effect showing unexpected higher efficacy.

βhCG can be coupled to tetanus toxoid (TT), diphtheria toxoid or promiscuous peptides using the hetero-bifunctional reagents succinimidyl 6-{3′-[2-pyridyldithio]-propionamido} hexanoate (SPDP) and/or succinimidyl 4-[N-maleimidomethyl]cyclohexane-1-carboxylate (SMCC).

In accordance with the present invention in one embodiment there is provided a therapeutic and/or prophylactic cancer vaccine composition for cancer therapy, wherein the composition comprises hCG, Mycobacterium w and a pharmaceutically acceptable excipient.

The vaccine composition as disclosed in the present invention is useful for the treatment and/or prevention of cancer selected from the group consisting of human lung cancer, colon cancer, testicular cancer, ovarian cancer, bladder cancer, renal cancer, prostate cancer, head and neck cancer and colorectal cancer.

In yet another embodiment of the present invention there is provided a therapeutic and/or prophylactic cancer vaccine composition for cancer therapy, wherein the composition comprises hCG, Mycobacterium w and a pharmaceutically acceptable excipients, wherein the hCG is αhBCG or βhCG.

In yet another embodiment of the present invention there is provided a therapeutic and/or prophylactic cancer vaccine composition for cancer therapy, wherein the composition comprises hCG, Mycobacterium w and a pharmaceutically acceptable excipients, wherein the hCG is αhBCG.

In yet another embodiment of the present invention there is provided a therapeutic and/or prophylactic cancer vaccine composition for cancer therapy, wherein the composition comprises hCG, Mycobacterium w and a pharmaceutically acceptable excipients, wherein the hCG is βhCG.

In yet another embodiment of the present invention there is provided a therapeutic and/or prophylactic cancer vaccine composition for cancer therapy, wherein the composition comprises hCG, Mycobacterium w and a pharmaceutically acceptable excipients, wherein the hCG is αhBCG and βhCG.

In another embodiment of the present invention there is provided a therapeutic and/or prophylactic cancer vaccine composition for cancer therapy, wherein the composition comprises hCG, Mycobacterium w and pharmaceutically acceptable excipients, wherein the hCG is conjugated with tetanus toxoid, diphtheria toxoid or T helper peptide.

In another embodiment there is provided T helper peptide that can be used for conjugation of hCG, wherein the T helper peptide is derived from a pathogen protein selected from the group consisting of tetanus toxin, Plasmodium falciparum circumsporozoite protein, respiratory syncytial virus 1A protein, measles virus fusion protein, influenza virus hemagglutinin and hepatitis B surface antigen.

In another embodiment of the present invention there is provided a therapeutic and/or prophylactic cancer vaccine composition for cancer therapy, wherein the composition comprises hCG, Mycobacterium w and a pharmaceutically acceptable excipients, wherein the Mycobacterium w is killed by physical method selected from the group consisting of heat radiation most preferably by heat in form of autoclaving.

In another embodiment of the present invention there is provided a therapeutic and/or prophylactic cancer vaccine composition for cancer therapy, wherein the composition comprises hCG, Mycobacterium w and pharmaceutically acceptable excipients, wherein the composition is administered to a subject in need thereof as a dose ranging from 10 to 500 million Mycobacterium w and 2 μg to 50 μg βhCG.

In another embodiment of the present invention there is provided a therapeutic and/or prophylactic cancer vaccine composition for cancer therapy, wherein the composition comprises hCG, Mycobacterium w, pharmaceutically acceptable excipients and adjuvant.

In another embodiment there is provided adjuvant selected from the group consisting of aluminum hydroxide, Incomplete Fruend's Adjuvant, endotoxin based adjuvants, mineral oil, mineral oil and surfactant, Ribi adjuvant, Titer-max, syntax adjuvant formulation, aluminum salt adjuvant, nitrocellulose adsorbed antigen, immune stimulating complexes, Gebru adjuvant, super carrier, elvax 40w, L-tyrosine, monatanide (manide-oleate compound), Adju prime, Squalene, Sodium phthalyl lipopoly saccharide, calcium phosphate, saponin and muramyl dipeptide (MDP).

In yet another embodiment of the present invention there is provided a therapeutic and/or prophylactic cancer vaccine composition for cancer therapy, wherein the composition comprises hCG, Mycobacterium w and pharmaceutically acceptable excipients, wherein the composition is administered to a subject in need thereof as a dose ranging from 10 to 500 million Mycobacterium w and 1 μg to 500 μg hCG.

In yet another embodiment of the present invention there is provided a therapeutic and/or prophylactic cancer vaccine composition for cancer therapy, wherein the composition comprises hCG, Mycobacterium w and pharmaceutically acceptable excipients, wherein the composition is administered to a subject in need thereof as a dose ranging from 10 to 500 million Mycobacterium w and 50 μg to 500 μg hCG.

In yet another embodiment of the present invention there is provided a therapeutic and/or prophylactic cancer vaccine composition for cancer therapy, wherein the composition comprises hCG, Mycobacterium w and pharmaceutically acceptable excipients, wherein the vaccine composition is administered to a subject in need thereof as a dose of 100 to 200 million Mycobacterium w and 100 to 200 μg hCG.

In yet another embodiment of the present invention there is provided a therapeutic and/or prophylactic cancer vaccine composition for cancer therapy, wherein the composition comprises hCG, Mycobacterium w and pharmaceutically acceptable excipients, wherein the vaccine composition is administered to a subject in need thereof as a dose of 100 million Mycobacterium w and 100 μg hCG.

In yet another embodiment of the present invention there is provided a therapeutic and/or prophylactic cancer vaccine composition for cancer therapy, wherein the composition comprises hCG, Mycobacterium w and pharmaceutically acceptable excipients, wherein the vaccine composition is administered to a subject in need thereof in combination with a therapy selected from the group consisting of radiation therapy and chemotherapy.

In yet another embodiment of the present invention there is provided a pharmaceutical preparation comprising an effective amount of hCG and Mycobacterium w.

In further embodiment of the present invention there is provided a method of treatment or prevention of cancer, wherein the method comprising administering to a subject in need thereof a vaccine composition comprising hCG and Mycobacterium w.

In further embodiment of the present invention there is provided a method of treatment or prevention of cancer, wherein the method comprising administering to a subject in need thereof a vaccine composition comprising hCG and Mycobacterium w, wherein the cancer is selected from the group of lung cancer cells, colon cancer cells, testicular cancer cells, ovarian cancer cells, bladder cancer cells, renal cancer cells, prostate cancer cells, head and neck cancer.

The vaccine composition as disclosed in the present invention is administered to a subject in need thereof as a dose ranging from 100 million to 500 million Mw bacteria and 100 to 500 μg hCG/hCG equivalent of the conjugate.

In still another embodiment of the present invention there is provided use of hCG and Mycobacterium w in combination for the preparation of a therapeutic and/or prophylactic composition for the treatment and or prevention of cancer.

In yet another embodiment of the present invention there is provided a method for treatment and/or prevention of cancer, the method comprises administering to a subject in need thereof an effective amount of the vaccine composition comprising hCG, Mycobacterium w and pharmaceutically acceptable excipients.

A method for treatment and/or prevention of cancer as disclosed in the present invention wherein the effective amount of the vaccine composition comprising hCG, Mycobacterium w and pharmaceutically acceptable excipients is administered to a subject in need thereof in combination with a therapy selected from the group consisting of radiation therapy and chemotherapy.

The method disclosed in the present invention, wherein the step of administering the effective amount of the vaccine composition to a subject in need thereof is performed either simultaneously or sequentially.

Following examples demonstrate the invention and are not limiting for purpose of invention.

### EXAMPLES

### Example 1

βhCG-TT Conjugation

βhCG was coupled to TT using sulfosuccinimidyl 6-{3′-[2-pyridyldithio]-propionamido} hexanoate (sulfo-LC-SPDP). Briefly, βhCG and TT were treated individually at 25° C. for 1 hr with SPDP. Unreacted SPDP was removed by gel filtration. Activated βhCG and TT were mixed in a ratio of 6 βhCG molecules to every TT and an incubation carried out for 24 hrs at 4° C. Unconjugated βhCG and TT were removed by gel filtration. The βhCG-TT conjugate was adsorbed on aluminium hydroxide.

Mycobacterium w Culture

Mycobacterium w were grown in Middlebrook 7H9 media supplemented with 10% albumin-dextrose complex enrichment, 0.02% glycerol and 0.05% Tween 80. Mycobacteria were harvested, washed thrice with phosphate buffered saline (PBS), re-suspended in PBS and then killed by autoclaving at 121° C. at a pressure of 15 lb/in² for 20 min.

### Example 2

### Use of Mycobacterium w for Anti-hCG Vaccination Showing Synergistic Effect

Six to eight week old BALB/c and C57BL/6 mice were intramuscularly immunized with 2 μg of the βhCG-TT conjugate adsorbed on aluminum hydroxide. One group of animals also received an intra-muscular injection of 10⁷ autoclaved Mycobacterium w. Each animal received a total of three injections at fortnightly intervals. Blood samples were withdrawn at weeks three and five. Anti-hCG antibodies in sera were estimated by radioimmunoassay and the neutralization capacity of the antibodies by radioreceptor assay. Antibody isotypes were determined by ELISA. Sera were also assessed for the presence of antibody reactivity towards COLO 205 and ChaGo cells by immunofluorescence.

Radioimmunoassays revealed that at both three and five weeks post-immunization, inclusion of Mycobacterium w significantly enhanced anti-hCG titres. In BALB/c animals, which respond relatively well to the prototypic vaccine, titres were further increased in the inclusion of the bacterium. C57BL/6 mice, on the other hand, are low responders to the prototypic vaccine; significantly, in these animals too, the inclusion of Mycobacterium w had a highly stimulatory effect on anti-hCG antibody levels. The effect was due to pure adjuvant properties of the bacterium and not due a fortuitous cross-reactivity between Mycobacterium w and hCG, since animals immunized with just Mycobacterium w demonstrated no anti-hCG titres in their serum. Elicited antibodies were of high affinity, as measured by cold displacement radio-immunoassays. In addition, antibodies had the capacity to prevent hCG-receptor interaction as assessed by radioreceptor assays. Inclusion of Mycobacterium w in the vaccine formulation led to significant increases in IgG2b responses, a fact indicative of a Th1 skew. Elicited antibodies in both strains of mice had the capacity to bind cell surface antigens of COLO 205 and Chago cells. Enhance reactivity was observed upon cell permeabilization.

### Example 3

### Use of Mycobacterium w Showing Synergistic Effect in the Immunotherapy of LL2 Lung Tumors in Mice

C57BL/6 animals were subcutaneously implanted with 10⁴ cells. Animals received three intra-muscular injections of the prototypic anti-hCG vaccine (βhCG-TT adsorbed on aluminum hydroxide) at monthly intervals. Each injection consisted of 2 μg gonadotropin equivalent. Two schedules were adopted: In the first schedule, tumor implantation and immunization were concurrent, while in the second, immunization was initiated sixty three days before tumor implantation. In both schedules, one vaccine treatment group additionally contained 10⁷ autoclaved Mycobacterium w, and one group was administered only Mycobacterium w. Serum antibody titres, as well as tumor incidence and volumes were measured at regular intervals. It was observed immunization with βhCG-TT alone (in both treatment schedules) led to significant decreases in tumor growth rates and incidence. Similar results were obtained in animals receiving Mycobacterium w alone. When βhCG-TT and Mycobacterium w were combined, significant improvements in efficacy were observed, both in terms of tumor incidence and volumes. In this case too, similar results were seen in both treatment schedules. These results indicate that while individual treatment with βhCG-TT or Mycobacterium w resulted in significant benefit, combination therapy provides synergistic benefits.

What is claimed is:

1. A vaccine composition for cancer therapy and/or cancer prophylaxis, the composition comprising hCG conjugate, Mycobacterium w and a pharmaceutically acceptable excipient.

2. The vaccine composition of claim 1, wherein the cancer is selected from the group consisting of human lung cancer, colon cancer, testicular cancer, ovarian cancer, bladder cancer, renal cancer, prostate cancer, head and neck cancer and colorectal cancer.

3. The vaccine composition of claim 1, wherein the hCG is βhCG.

4. The vaccine composition of claim 1, wherein the hCG is conjugated with tetanus toxoid, diphtheria toxoid, or T helper peptide.

5. The vaccine composition of claim 4, wherein the T helper peptide is derived from a pathogen protein selected from the group consisting of tetanus toxin, Plasmodium falciparum circumsporozoite protein, respiratory syncytial virus 1A protein, measles virus fusion protein, influenza virus hemagglutinin, and hepatitis B surface antigen.

6. The vaccine composition of claim 1, wherein the Mycobacterium w is killed by physical method of heat radiation.

7. The vaccine composition of claim 6, wherein the heat radiation comprises heat by autoclaving.

8. The vaccine composition of claim 1, the vaccine composition optionally comprising an adjuvant selected from the group consisting of aluminum hydroxide, Incomplete Fruend's Adjuvant, endotoxin based adjuvants, mineral oil, mineral oil and surfactant, Ribi adjuvant, Titer-max, syntax adjuvant formulation, aluminum salt adjuvant, nitrocellulose adsorbed antigen, immune stimulating complexes, Gebru adjuvant, super carrier, elvax 40w, L-tyrosine, monatanide (manide-oleate compound), Adju prime, Squalene, Sodium phthalyl lipopoly saccharide, calcium phosphate, saponin, and muramyl dipeptide (MDP).

9. The vaccine composition of claim 1, wherein the composition is administered to a subject in need thereof at a dose ranging from 10 to 500 million Mycobacterium w and 1 μg to 50 μg hCG.

10. The vaccine composition of claim 1, wherein the composition is administered to a subject in need thereof at a dose ranging from 10 to 500 million Mycobacterium w and 50 μg to 500 μg hCG.

11. The vaccine composition of claim 1, wherein said vaccine composition is administered to a subject in need thereof at a dose of 100 to 200 million Mycobacterium w and 100 to 200 μg hCG.

12. The vaccine composition of claim 1, wherein the vaccine composition is administered to a subject in need thereof at a dose of 100 million Mycobacterium w and 100 μg hCG.

13. The vaccine composition of claim 1, wherein the vaccine composition is administered to a subject in need thereof in combination with a therapy selected from the group consisting of radiation therapy and chemotherapy.

14. The vaccine composition of claim 1, wherein the vaccine composition is administered by parental route, intramuscular, subcutaneous, or intradermal route.

15. A vaccine composition, comprising hCG conjugate, Mycobacterium w and a pharmaceutically acceptable excipient, wherein the vaccine composition stimulates a non-specific immune response in a subject to restrict tumor growth in the subject.

16. A method of treating cancer in a subject, comprising administering a vaccine composition comprising hCG conjugate, Mycobacterium w and a pharmaceutically acceptable excipient to the subject.

17. The method of claim 16, wherein the cancer is selected from the group consisting of human lung cancer, colon cancer, testicular cancer, ovarian cancer, bladder cancer, renal cancer, prostate cancer, head and neck cancer and colorectal cancer.

18. The method of claim 16, further comprising administering an adjuvant selected from the group consisting of aluminum hydroxide, Incomplete Fruend's Adjuvant, endotoxin based adjuvants, mineral oil, mineral oil and surfactant, Ribi adjuvant, Titer-max, syntax adjuvant formulation, aluminum salt adjuvant, nitrocellulose adsorbed antigen, immune stimulating complexes, Gebru adjuvant, super carrier, elvax 40w, L-tyrosine, monatanide (manide-oleate compound), Adju prime, Squalene, Sodium phthalyl lipopoly saccharide, calcium phosphate, saponin, and muramyl dipeptide (MDP).

19. The method of claim 16, wherein the vaccine composition non-specifically stimulates an immune response in the subject to independently restrict tumor growth.
